(19) United States
(12) Patent Application Publication
(10) Pub. No.: US 2011/0009722 A1
(43) Pub. Date: Jan. 13, 2011

(54) Historical Trend Icons For Physiological Parameters

(75) Inventors: Scott Amundson, Oakland, CA (US); Li Li, Milpitas, CA (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/743,465

(22) PCT Filed: Dec. 24, 2008
(86) PCT No.: PCT/US08/88310

§ 371 Date: Aug. 30, 2010

Related U.S. Application Data
(60) Provisional application No. 61/009,101, filed on Dec. 26, 2007.

(51) Int. Cl. A61B 5/1455; G06F 3/048

(52) U.S. Cl. 600/324; 715/810

(57) ABSTRACT

Embodiments relate to systems and methods for displaying graphical icons representing a detected medical condition or a sensor fault. Specifically, embodiments of relate to a monitoring system that includes a sensor configured to obtain a physiologic signal from a patient, and a monitor communicatively coupled to the sensor and configured to receive the signal. In an embodiment, the monitor includes a processor adapted to compute physiological data based on the signal and identify a pattern in the physiological data, wherein the pattern relates to a condition of the patient, the sensor, or the monitor. In an embodiment, the processor also selects a graphical icon indicative of the pattern and provides the selected icon to a display.

### BACKGROUND

The present disclosure relates generally to medical devices, and more specifically to medical devices capable of measuring and displaying physiological parameters.

This section is intended to introduce the reader to various aspects which may be related to various aspects of the present disclosure, which are described and/or claimed below. This discussion is believed to be helpful in providing the reader with background information to facilitate a better understanding of the various aspects of embodiments. Accordingly, it should be understood that these statements are to be read in this light, and not as admissions of prior art.

In the field of medicine, doctors often desire to monitor certain physiological characteristics of their patients. Accordingly, a wide variety of devices may have been developed for monitoring many such characteristics of a patient. Such devices may provide doctors and other healthcare personnel with the information they may utilize to provide care for their patients. As a result, such monitoring devices may have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient.

Pulse oximeters typically utilize a patient monitoring device that computes physiological parameters and displays information related to the various physiological parameters calculated. The information related to one physiological parameter is typically presented to a clinician in the form of numerical data and a graph showing oxygen saturation (SpO₂) as a function of time. The SpO₂ graph may represent data recorded over several minutes or hours. Due to display size constraints and the volume of data, the historical SpO₂ graph may be too long to appear on the display in its entirety. Therefore, the clinician may be required to scroll through several screens in order to observe the SpO₂ trending data to ascertain a patient's condition. This process can be complex and time consuming, as the clinician scrolls through hours of collected data. In many cases, there may be too much data for a clinician to process quickly and accurately, because a particular pattern or trend may not be visible on a single screen. Additionally, nurses and technicians who have frequent contact with a patient may not have the knowledge and experience necessary to determine a patient's condition from observing the historical SpO₂ data. Typically, a specialist, who generally may have significantly less contact with the patient, is required to interpret this data.

### SUMMARY

Certain aspects commensurate in scope with the embodiments are set forth below. It should be understood that these aspects are presented merely to provide the reader with a brief summary of certain forms the embodiments might take and that these aspects are not intended to limit the scope of the disclosure.

In an embodiment, there is provided a monitoring system that includes a sensor configured to obtain a physiologic signal from a patient. The system may also include a monitor configured to receive the signal from the sensor. In an embodiment, the monitor includes a processor adapted to compute physiological data based on the generated signals, identify a pattern in the physiological data, wherein the pattern relates to a condition of the patient, the sensor, or the monitor, select a graphical icon indicative of the pattern, and provide the selected icon to a display.

In an embodiment, there is also provided a tangible machine readable medium comprising code for computing physiological data based on received electrical signals and recognizing one or more patterns in the physiological data, the one or more patterns indicating a medical condition or sensor fault. In an embodiment, the tangible machine readable medium includes code for correlating the patterns to a graphical icon representative of the recognized pattern; and code for outputting the graphical icon to a graphical display.

### BRIEF DESCRIPTION OF THE DRAWINGS

Advantages of the embodiments may become apparent upon reading the following detailed description and upon reference to the drawings in which:

FIG. 1 illustrates a block diagram of a patient monitoring system, such as a pulse oximeter, in accordance with an embodiment;

FIG. 2 is a view of an embodiment of a display of the patient monitoring system of FIG. 1 that is adapted to display either a graph or an icon related to the graph, according to an embodiment;

FIG. 3 illustrates an exemplary plot of a percent oxygen saturation of hemoglobin over a fifteen minute period, according to an embodiment;

FIG. 4a illustrates a graphical icon indicating the detection of airway instability in accordance with an embodiment;

FIG. 4b illustrates a graphical icon indicating the detection of sleep apnea in accordance with an embodiment;

FIG. 4c illustrates a graphical icon representing consistent, normal oxygen saturation in accordance with an embodiment;

FIG. 4d illustrates a graphical icon representing a steady decline in the percent oxygen saturation of hemoglobin in accordance with an embodiment;

FIG. 4e illustrates a graphical icon representing a sudden drop in the percent oxygen saturation of hemoglobin in accordance with an embodiment;

FIG. 4F illustrates a graphical icon representative of sensor signal interference in accordance with an embodiment; and

FIG. 5 is a process flow diagram for the operation of the exemplary patient monitoring system, wherein the diagram depicts that the system is configured to detect medical conditions or sensor faults and to display graphical icons representative of the detected medical conditions or sensor faults in accordance with an embodiment.

### DETAILED DESCRIPTION

Various embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Significant research may be being performed to develop systems which automatically detect a patient's condition based on physiological data, such as historical SpO₂ trending information, gathered by medical devices. Once a particular condition may be detected, some systems may be configured to provide an audible and/or visual warning. For example, if the system determines a patient's SpO₂ level drops below a specified value for a requisite period of time, an audible alarm may sound to indicate such an occurrence. However, the use of an audible alarm may be of limited use as it may not provide the clinician with any useful information beyond the fact that a condition, i.e., low blood oxygen saturation, has been detected.

One addition to the use of an audible alarm may include displaying text on a display of the medical device to indicate the type of condition that has occurred. This text may be generated automatically based on a computer analysis of historical data. For example, if a pulse oximeter determined that a patient's airway is unstable based on historical SpO₂ data, it may display the message “AIRWAY INSTABILITY” on the screen. However, text may be difficult to read from a distance. Additionally, the manufacturer of the pulse oximeter configured to display the text may be required to translate the message into several different languages depending on the market. The present disclosure may address these issues by providing an unambiguous way to indicate a patient's condition to a clinician without the issues associated with displaying text or manually analyzing historical data.

Specifically, the present disclosure may relate to a medical device configured to display a graphical icon indicative of a medical condition, a historical trend, a hardware fault, a monitor error or a sensor fault, for example. In an embodiment, the medical device is a pulse oximeter programmed to recognize a medical condition, a monitor error, or sensor fault based on preprogrammed patterns that may be recognized in the historical data. Once the pulse oximeter recognizes a particular pattern, it displays a graphical icon indicative of the condition correlated to the pattern. The graphical icon may appear similar to a historical trend it represents and may allow the clinician to quickly, easily and accurately identify the patient's condition. Specifically, as will be discussed in greater detail below, the icon may include characteristics of the historical trend it represents. Upon notification of the condition via the icon, a clinician may take appropriate action, which may include following a particular course of treatment for the patient or adjusting the positioning of the sensor, for example.

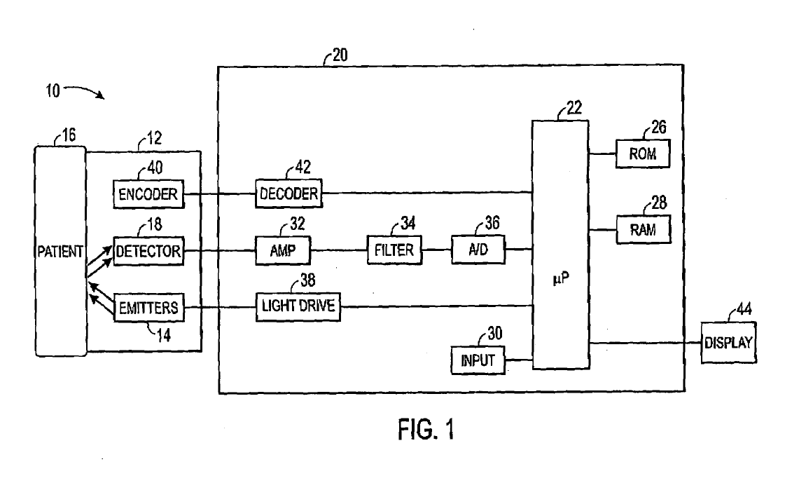

Turning to FIG. 1, a block diagram of a pulse oximeter is illustrated in accordance with an embodiment and is generally designated with the reference number 10. One exemplary monitor is the Model N600x available from Nellcor Purtain Bennett LLC. In an embodiment, the pulse oximeter 10 includes a sensor unit 12 having an emitter 14 configured to transmit electromagnetic radiation, i.e., light, into the tissue of a patient 16. In an embodiment, the emitter 14 may include a plurality of LEDs operating at discrete wavelengths in the red and infrared portions of the electromagnetic radiation spectrum. In an embodiment, the emitter 14 may also be a broad spectrum emitter, such as a white light source, for example.

In an embodiment, a photoelectric detector 18 in the sensor 12 may be configured to detect the scattered and reflected light and to generate an electrical signal, e.g., current, corresponding to the detected light. The sensor 12 may direct the detected signal from the detector 18 to a monitor 20, which processes the signal and calculates various physiological parameters.

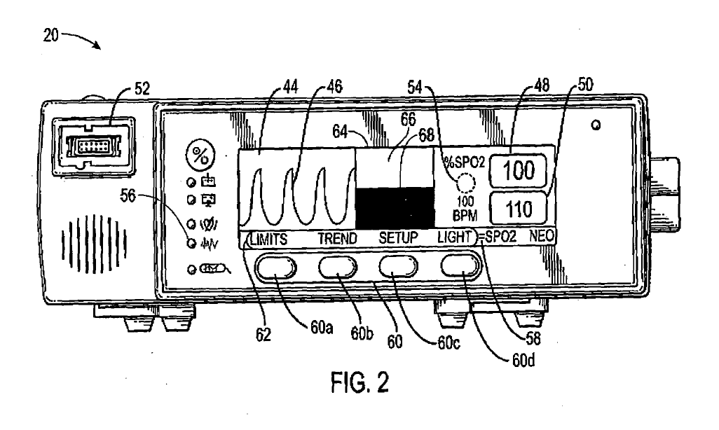

In an embodiment, the monitor 20 includes a microprocessor 22, which is configured to calculate physiological parameters using algorithms programmed into the monitor 20. The microprocessor 22 may be connected to other component parts of the monitor 20, such as a ROM 26, a RAM 28, and other inputs 30. The ROM 26 may be configured to store the algorithms used to compute physiological parameters. The RAM 28 may be capable of storing the values detected by the detector 18 for use in the algorithms. In an embodiment, the inputs 30 may allow a user, such as a clinician, to interface with the monitor 20. Specifically, as will be described in greater detail with regard to FIG. 2 below, the inputs 30 may allow for a clinician to scroll through screens of historical data or select item from a menu.

In an embodiment, in the monitor 20, the signals may be amplified and filtered by amplifier 32 and filter 34, respectively, before being converted to digital signals by an analog-to-digital converter 36. Once digitized, the signals may be used to calculate the physiological parameters and/or may be stored in RAM 28.

In an embodiment, a light drive unit 38 in the monitor 20 may be capable of controlling the timing of the emitters 14. While the emitters 14 are manufactured to operate at one or more certain wavelengths, variances in the wavelengths actually emitted may occur which may result in inaccurate readings.

In an embodiment, to help avoid inaccurate readings, an encoder 40 and decoder 42 may be used to calibrate the monitor 20 to the actual wavelengths being used. The encoder 40 may be a resistor, for example, whose value corresponds to coefficients used in algorithms for computing the physiological parameters. Alternatively, the encoder 40 may be a memory device, such as an EPROM, that stores wavelength information and/or the corresponding coefficients. In an embodiment, once the coefficients are determined by the monitor 20, they are inserted into the algorithms in order to calibrate pulse oximeter 10.

In an embodiment, once the physiological parameters are calculated, monitor 20 may be configured to display the calculated parameters on a display 44. FIG. 2 illustrates such a monitor 20 in accordance with an embodiment. In an embodiment as illustrated, the monitor 20 includes the display 44 to display computed physiological data and other information. In other embodiments, the monitor 20 may output a signal to a separate display device, such as a liquid crystal display located near the monitor 20, for example. In yet another embodiment, a signal may be transmitted via a network to a display located remotely from the monitor 20. For example, the display may be a display on a personal digital assistant (PDA) or other portable computing device, so that if the PDA is in the possession of a clinician, the clinician may be informed of the patient's condition while at a location remote from the monitor 20.

The display 44 may be configured to display a plethysmographic waveform 46, a percent oxygen saturation 48, and/or a pulse rate 50, and/or combinations thereof. The oxygen saturation may be a functional arterial hemoglobin oxygen saturation measurement in units of percentage SpO₂, and the pulse rate 50 may indicate a patient's pulse rate in beats per minute.

The monitor 20 may also display information related to alarms and monitor settings. For example, in some embodiments, the monitor 20 may employ SatSeconds™ by Nellcor™ to detect alarms and manage nuisance alarms. SatSeconds™ may include activation of an alarm based on limits that may include the integral of time and depth of a desaturation event and may also include an indicator 54 that may serve to inform the operator that an SpO₂ reading has been detected outside of the limit settings. The monitor may also include other settings relating to signal quality, such as a signal quality indicator light 56. The display 44 may also include an alarm status indicator (not shown), and special settings such as a fast response mode setting indicator 58.

To facilitate user input, the monitor 20 may include a number of keys 60 that may correlate to the input 30 of FIG. 1. In an embodiment, the keys 60 may be related to the operating functions. The keys 60 may include fixed function keys and programmable function keys, along with associated key icons in a soft key menu 62. In other words, the four keys 60a, 60b, 60c, and 60d are pressed to select a corresponding one of the soft key icons. The soft key menu 62 indicates which software menu items can be selected through the keys 60. Pressing a key 60 associated with, or adjacent to, an icon in the icon menu 62, selects the option.

In an embodiment, the display 44 may be configured to display a graphical icon 64 indicative of a detected condition, historical trend or a sensor fault. The graphical icon 64 may be displayed in conjunction with a plethysmographic waveform 46 (as shown) or independently from the plethysmographic waveform 46. Alternatively, the graphical icon 64 may be displayed with a historical trend of oxygen saturation (not shown). Additionally, in an embodiment, the graphical icon 64 may be displayed intermittently in place of the plethysmographic waveform 46 when a particular condition is detected.

The use of a graphical icon 64 may simplify and expedite the diagnosis of a particular condition or bring a particular condition to the attention of a clinician. As discussed above, the volume of physiological data to sort through to find a particular trend and the complex patterns that may indicate particular conditions may make it difficult for a clinician to accurately and quickly diagnose potential issues. The follow-discussion describes the development of the icons that suggest a particular condition.

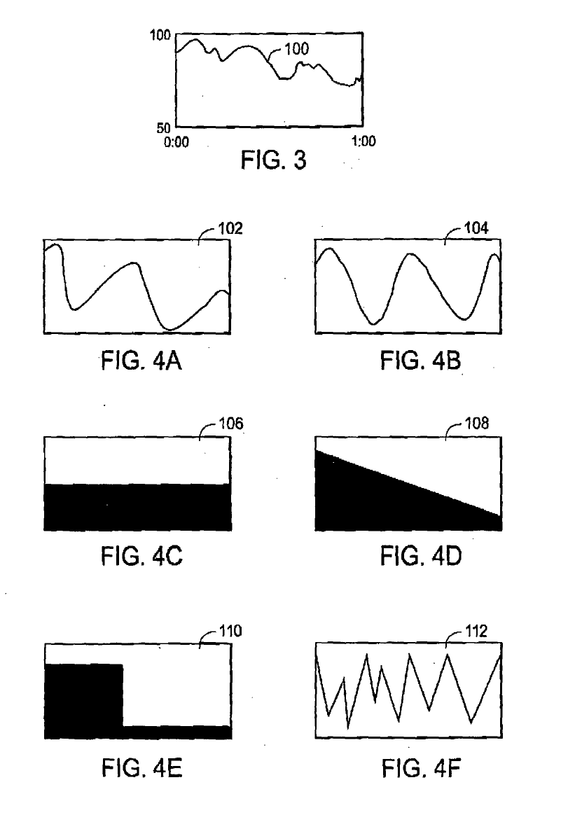

Referring to FIG. 3, an embodiment of a percent oxygen saturation trace 100 over the course of 15 minutes is shown. The x-axis represents time and the y-axis represents the percent saturation of oxygen (SpO₂). As can be seen, the trace 100 contains peaks and valleys, and exhibits a general downward trend. Analysis by a trained clinician may reveal that the trace 100 of the percent oxygen over the course of the hour indicates a particular medical condition, such as airway instability. However, to an untrained user, the trace 100 may carry little or no significance. As such, in accordance with the present disclosure, the monitor 20 may be configured to evaluate the percent oxygen data and compare the data with known patterns of airway instability to detect if the data that defines the trace 100 indicates airway instability.

Once it has determined that the data indicates airway instability, the monitor 20 may display a graphical icon representative of airway instability, such as the graphical icon 102 shown in FIG. 4a, for example. The graphical icon 102 conveys the idea of air instability by showing and/or exaggerating important characteristics of airway instability that may or may not be evident in a SpO₂ graph. In this embodiment, the icon 102 looks somewhat similar to the trace 100 in that the graphical icon 102 includes the peaks, valleys and general downward trend. Thus, the graphical icon 102 may look similar to an actual plot of the SpO₂ for a patient experiencing airway instability. In other embodiments, however, where a data trace may not indicate airway instability so readily, the graphical icon 102 may not look similar to the actual plot of SpO₂ values. In fact, because the icon 102 provides a clear indication of airway stability in cases where the actual data trace may be difficult to decipher, the use of a graphical icon, such as graphical icon 102, may be particularly useful.

The similarity of a particular icon to a graph of an actual medical condition or sensor fault may result in ease of training on use of the graphical icons. Specifically, a clinician trained in identifying medical conditions and sensor faults from a graph may easily recognize the significance or meaning of an icon because the icon would mimic, to some extent, the graph of the represented condition. In addition, other caregivers, not trained to identify patterns in a graph, could be easily trained to recognize the simplistic icons representative of a particular condition. Additionally, a legend or key for the graphical icons may be provided with the monitor 20 so that the icons could easily be recognized as representing a particular condition. Thus, virtually anyone may identify a medical condition or sensor fault from a cursory view of an icon displayed on the monitor's display 44.

Graphical icons may be developed and used to identify a wide variety of conditions besides airway instability. Specifically, in addition to airway instability, the monitor 20 may be configured to determine the presence of apneaic events (or sleep apnea), stable saturation, slow desaturation, or rapid desaturation. Additionally, graphical icons may be developed to indicate sensor errors. FIGS. 4b, 4c, 4d, and 4f each illustrate example graphical icons related to these several conditions.

For example, FIG. 4b illustrates an embodiment of a graphical icon 104 which may indicate sleep apnea. As can be seen, the graphical icon 104 shows high peaks and sharp drops to low valleys to mimic characteristics of an actual plot of sleep apnea. FIG. 4c illustrates an exemplary graphical icon 106 for a stable condition. The image depicted by this graphical icon 106 may be a solid rectangle covering the bottom half of the icon, as shown. In another embodiment (not shown), the maintenance of normal oxygen saturation levels may be represented by a horizontal line that extends across the middle of the icon.

An embodiment of a graphical icon 108 representing a slow desaturation is illustrated in FIG. 4d. As illustrated, the graphical icon 108 may be a nearly solid right triangle with a downward slope on the hypotenuse to indicate the desaturation. In another embodiment (not shown), steady or slow desaturation may be represented by a single line with a downward slope. In an embodiment, the slope of the line may depend on the rate of desaturation. For example, the faster the desaturation, the steeper the slope. For example, FIG. 4e illustrates an embodiment of a graphical icon 110 to indicate rapid desaturation, where the rapid desaturation event is represented by a vertical drop from a high level to a lower level. In another embodiment, the drop may be represented by a curve which demonstrates an instantaneous drop in SpO₂.

In an embodiment, the decision point for distinguishing between a steady decline in SpO₂ levels and a rapid or instantaneous desaturation may be based at least in part upon the size of the sampling window and the degree of desaturation. In an embodiment, in order to determine that there has been a slow and steady desaturation occurrence, the monitor 20 may need to evaluate the SpO₂ history for several hours. However, in the event of a rapid or instantaneous desaturation event, the monitor may evaluate only an instantaneous reading where the saturation levels dropped a significant amount, such as, 10-20 percent, for example.

In some situations a sudden drop in saturation levels as measured by the monitor 20 may not always indicate an actual desaturation event. For example, the sensor 12 may be poorly coupled, or entirely uncoupled, to a patient's tissue. In such a case, the sensor is unable to accurately detect the amount of light that has passed through tissue and, as such, the monitor 20 cannot provide accurate calculations of physiological parameters.

Additionally, other events may induce noise in the sensor signal that makes accurate calculations difficult. Although a historical trend may indicate that there have been large, rapid changes in SpO₂ data as a function of time, such changes may be due to interference, sensor errors, or monitor errors and not to the patient's actual physical status. Accordingly, patterns related to interference, sensor errors or monitor errors may be recognized.

FIG. 4f illustrates an embodiment of a graphical icon 112 for signal interference. As illustrated, the graphical icon 112 may be representative of signal interference and may include a jagged line to indicate that the interference is random, significant and not a physiological pattern. One example of a monitor error or hardware error that may be detected is signal drop out due to a connector pin being loose. Because the loose pin may result in a signal dropping out suddenly and then returning, an icon for detecting such an occurrence may resemble the icon 112 of FIG. 4f.

Other conditions which could activate an alarm may include, but are not limited to a weak signal, sensor off, sensor disconnect, bad sensor placement, arrhythmia, venous pulsation (sensor placed over a pulsating vein instead of an artery), sensor motion, EMI/RFI (electromagnetic or radio frequency interference). It will be appreciated that various icons, and/or indicators may b utilized to indicate these and other conditions.

It will be appreciated that this disclosure is not limited to the conditions which may be indicated to a clinician to alert them. Furthermore, the disclosure is not limited to the type, style, and particular form of the graphical icon.

In an embodiment, the graphical icons 102, 104, 106, 108, 110, and 112 may be represented in a monochromatic format or, alternatively, in color. A monochromatic display may provide cost savings, however, a color display may permit additional functionality to be integrated in the graphical icon system. In an embodiment, the color of the graphical icons may be correlated with the severity of the condition. As such, a particular icon may represent multiple degrees of severity depending upon its color, with green representing a slight condition, yellow representing a moderate condition, and red representing a severe condition, for instance.

In an embodiment, particular colors may be correlated with the icons to further increase the ability to recognize a particular icon as representing the severity of a particular condition. In an embodiment, the rapid desaturation icon 110 could be presented in red as it would be a severe condition, while the sleep apnea icon 104 could be represented in yellow as a moderate condition and the stable icon 106 could be represented in green as a normal condition. Additionally, the background 66 and/or the foreground portions of the graphical icons may be used with the icon coloring techniques. Furthermore, the entire display may flash, in different colors to indicate the severity of an alarm condition.

In another embodiment, the color of the graphical icon 102 representing airway instability 102 may be correlated with the severity of the airway instability. In an embodiment, a patient may experience various degrees of airway instability which may be determined by the monitor 20. For a mild degree of instability, the graphical icon 102 representing the airway instability may appear yellow indicating that the condition exists but may not critically affect the health of the patient. For a greater degree of instability, the background color may be orange, and for a critical condition, the background and/or foreground color may be red. Thus, the use of colors may allow the clinician to readily determine the severity of the patient's condition.

Additionally, as mentioned above, the colors may be correlated with a particular detected event. The graphical icon 112 that represent sensor faults or interference may also be assigned a color to further differentiate their status as sensor faults and not medical conditions. Moreover, the color of the background 66 of the graphical icons may vary while the color of the foreground 68 may remain constant. In an embodiment, the foreground 68 of all graphical icons may be black. Alternatively, for example, the foreground 68 for airway instability graphical icon may be blue, while the foreground 68 for a graphical icon representative of sleep apnea may be green.

In an embodiment, the background 66 and/or foreground 68 of the graphical icon may flash to indicate severity and to draw attention to the graphical icon. The flashing may serve to alert the clinician to the critical nature of a particular medical condition. Furthermore, other audible or visual indications of a medical condition or sensor fault could accompany the displaying of the graphical icon to aid in alerting the clinician. For example, an audible alarm could sound when a particular condition is detected. The alarm may call the attention of the clinician to the display 44 to identify the detected condition by observing the graphical icon 64.

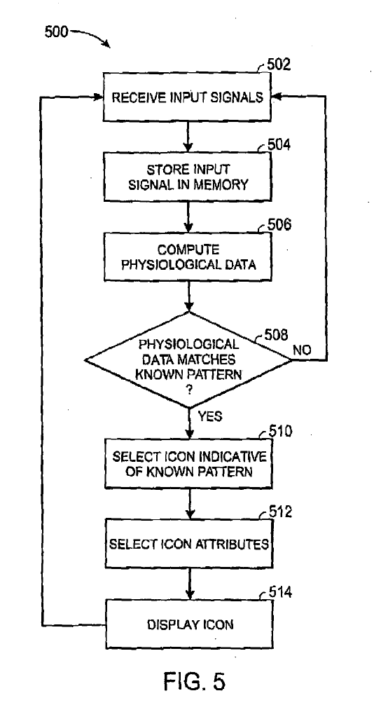

Referring to FIG. 5, a process flow diagram of a technique 500 of operating the medical device 10 in accordance with an embodiment is illustrated. The technique 500 may be in the form of software encoded on the ROM 26 and executed by the microprocessor 22, but those skilled in the art will recognize that software, firmware and/or hardware may be used.

In an embodiment, at block 502, a signal is received from a sensor 12. In the embodiment, the signal may be stored in a memory 28 of a monitor 20, as indicated at block 504. As discussed previously, the signal may be used by the monitor 20 to generate data in accordance with known algorithms, the data being representative of physiological data, as indicated at block 506.

In an embodiment, the computed physiological data may be compared to known patterns, as indicated at block 508. The patterns may be stored in the ROM 26 or the data may simply be analyzed to identify certain patterns. These patterns may include time histories of SpO₂ data, plethysmographic data, heart rate data, etc., for example, indicative of medical conditions or sensor faults. As discussed above, patterns for comparison include patterns which can be found in the physiological data which indicate airway instability, sleep apnea, consistent and acceptable oxygen saturation, steady or slow desaturation, rapid desaturation, signal interference and/or weak signal strength, as discussed above. If there is no pattern discernable by the monitor 20, then input signals are again received from a sensor 12 and the process starts over, as indicated by the line between block 508 and block 502.

However, if it is determined that the physiological data matches a particular known pattern indicative of a medical condition or a sensor fault, an icon is selected representative of the identified pattern, as indicated by block 510. In an embodiment, if the signal stored in the memory matches a pattern characteristic of sleep apnea, the monitor 20 may select an icon representative of that medical condition. As discussed above, the icon may depict an image that looks similar to a time history of SpO₂ data indicative of sleep apnea.

Next, the monitor 20 may determine the attributes of the selected icon, as indicated at block 512. In an embodiment, if the medical condition is relatively minor, the monitor 10 may select a yellow background color. Similarly, if the medical condition is critical, the monitor 20 may select a red background color. These colors may help the clinician identify the severity of the detected medical condition or sensor fault. In addition, the monitor 20 may determine that the icon should flash to further bring the patient's condition to the attention of the clinician. Finally, the monitor 10 may present the icon and any attribute on a display, as indicated by block 514. The method 500 may repeat for the duration of the operation of the monitor 20.

While the disclosure may allow for various modifications and alternative forms, embodiments have been shown by way of example in the drawings and were described in detail herein. However, it should be understood that the disclosure is not intended to be limited to the particular forms disclosed. Rather, the disclosure is to cover all modifications, equivalents and alternatives falling within the spirit and scope of the invention as defined by the following claims.

What is claimed is:

1. A monitoring system, comprising:
a sensor capable of obtaining a physiologic signal from a patient;
a monitor communicatively coupled to the sensor and capable of receiving the signal, the monitor comprising a processor capable of:
computing physiological data based on the signal;
identifying a pattern in the physiological data, wherein the pattern relates to a condition of the patient, the sensor, or the monitor;
selecting a graphical icon indicative of the pattern; and
displaying the selected icon to a display.

2. The monitoring system of claim 1, wherein the pattern comprises a pattern indicative of airway instability, sleep apnea, substantially normal oxygen saturation level, slow oxygen desaturation, rapid oxygen desaturation, signal interference, weak signal, sensor off, sensor disconnect, bad sensor placement, arrhythmia, venous pulsation, sensor motion, and/or electromagnetic or radio frequency interference, and/or combinations thereof.

3. The monitoring system of claim 1, wherein the pattern comprises a trend of SpO₂ data representative of airway instability and the graphical icon depicts characteristics of a generalized trend plot of SpO₂ data representative of airway instability.

4. The monitoring system of claim 1, wherein the pattern comprises a trend of SpO₂ data representative of sleep apnea and the graphical icon depicts characteristics of a generalized trend plot of SpO₂ data indicative of sleep apnea.

5. The monitoring system of claim 1, wherein the pattern comprises a trend plot of SpO₂ data representative of a substantially normal oxygen saturation level and the graphical icon depicts characteristics of a generalized trend plot of SpO₂ data representative of a substantially normal oxygen saturation level.

6. The monitoring system of claim 1, wherein the pattern comprises a trend plot of SpO₂ data representative of slow oxygen desaturation and the graphical icon depicts characteristics of a generalized trend plot of SpO₂ data representative of a slow oxygen desaturation.

7. The monitoring system of claim 1, wherein the pattern comprises a trend of SpO₂ data representative of rapid oxygen desaturation and the graphical icon depicts characteristics of a generalized trend of SpO₂ data representative of rapid oxygen desaturation.

8. The monitoring system of claim 1, wherein the pattern comprises a trend of SpO₂ data representative of signal interference and the graphical icon depicts general characteristics of signal interference.

9. The monitoring system of claim 1, wherein the pattern comprises a trend of SpO₂ data representative of a weak signal and the graphical icon depicts general characteristics of a weak signal.

10. The monitoring system of claim 1, wherein the processor is capable of assigning a color to the graphical icon based on the pattern.

11. The monitoring system of claim 1, wherein the processor is capable of selecting a color of the graphical icon based on a severity associated with the pattern.

12. A method comprising:
computing one or more physiological parameters of a patient;
identifying a pattern in the physiological parameters indicative of a medical condition or a sensor fault;
selecting a graphical icon corresponding to the pattern, the graphical icon generally indicating that the medical condition or sensor fault has been detected; and
displaying the graphical icon.

13. The method of claim 12 comprising determining a severity of the medical condition or sensor fault, and providing attributes to the graphical icon based on the determined severity.

14. The method of claim 13 wherein providing attributes comprises providing a color scheme to indicate severity.

15. The method of claim 12, wherein the pattern comprises a pattern indicative of airway instability, sleep apnea, substantially normal oxygen saturation level, slow oxygen desaturation, rapid oxygen desaturation, signal interference, weak signal, sensor off, sensor disconnect, bad sensor placement, arrhythmia, venous pulsation, sensor motion, and/or electromagnetic or radio frequency interference, and/or combinations thereof.

16. The method of claim 12, wherein the pattern comprises a trend of SpO₂ data representative of airway instability and the graphical icon depicts an image having generalized characteristics of the trend of SpO₂ data representative of airway instability.

17. The method of claim 12, wherein the pattern comprises a trend of SpO₂ data representative of sleep apnea and the graphical icon depicts an image having generalized characteristics of the trend of SpO₂ data representative of sleep apnea.

18. The method of claim 12, wherein the pattern comprises a trend of SpO₂ data representative of consistent and acceptable oxygen saturation and the graphical icon depicts an image having generalized characteristics of the trend of SpO₂ data representative of consistent and acceptable oxygen saturation.

19. The method of claim 12, wherein the pattern comprises a trend of SpO₂ data representative of steady or slow oxygen desaturation and the graphical icon depicts an image having generalized characteristics of to the trend of SpO₂ data representative of steady or slow oxygen desaturation.

20. The method of claim 12, wherein the pattern comprises a trend of SpO₂ data representative of rapid oxygen desaturation and the graphical icon depicts an image having generalized characteristics of the trend of SpO₂ data representative of rapid oxygen desaturation.

21. The method of claim 12, wherein the pattern comprises a trend of SpO₂ data representative of signal interference and the graphical icon depicts an image having generalized characteristics of the trend of SpO₂ data representative of signal interference.

22. The method of claim 12, wherein the pattern comprises a trend of SpO₂ data representative of a weak signal and the graphical icon depicts an image having generalized characteristics of the trend of SpO₂ data representative of a weak signal.

23. An article comprising: a tangible machine readable medium having stored thereon instructions that if executed, result in performance of a method comprising:
computing physiological data based on signals received from a patient;
recognizing a pattern in the physiological data, the pattern indicating a medical condition or sensor fault;
correlating the recognized pattern to a respective graphical icon representative of the recognized pattern; and
outputting the graphical icon to a graphical display.

24. The article of claim 23 comprising:
determining a level of severity indicated by the one or more patterns; and
providing attributes to the output graphical icon according to the level of severity.

25. The article of claim 23 comprising initiating an audible alarm in conjunction with the output graphical icon.

26. The article of claim 23 comprising alternating the display of the output graphical icon with display of the physiological data.
